(19) United States
(12) Patent Application Publication
(10) Pub. No.: US 2005/0096614 A1
(43) Pub. Date: May 5, 2005

(54) Cover layer for an absorbent article

(76) Inventors: Roberto Carlos Perez, Mexico City (MX); Juan Diego Tomas, Mexico City (MX); Elena Cristina Gonzalez, Mexico City (MX); Jaime Braverman, Atlanta, GA (US)

Correspondence Address: KIMBERLY-CLARK WORLDWIDE, INC., 401 NORTH LAKE STREET, NEENAH, WI, 54956, US

(21) Appl. No.: 10/696,181

(22) Filed: Oct. 29, 2003

(51) Int. Cl. A61F 13/15; A61F 13/20

(52) U.S. Cl. 604/378

(57) ABSTRACT

In one embodiment, a feminine care product comprises a cover layer containing a cover material and indicia imprinted thereon, a liquid impervious bottom layer bonded to the cover layer, and an absorbent core intermediate the cover layer and the bottom layer. The indicia is formed from an ink comprising a surfactant and/or a botanical extract in an amount effective to increase the absorptive properties of bodily exudates into the cover layer as compared to the cover layer without the surfactant and/or botanical extract.

### BACKGROUND

This disclosure generally relates to absorbent articles, and more particularly, relates to a topsheet or cover material for absorbent articles having indicia imprinted thereon.

Absorbent articles generally comprise a liquid permeable cover layer having a user-facing surface; a liquid impermeable bottom layer having an outer facing surface; and an absorbent core located intermediate between the cover and bottom layers. Typically, the cover layer is a polymeric material that is generally hydrophobic, which can deleteriously reduce the ease with which bodily fluids and exudates may be accepted into the absorbent core. There are several types of cover layers, such as woven fabrics, non-woven fabrics, reticulated films, polymer nets, and the like, as well as combinations of them, that are currently in use for various applications such as, for example, personal care absorbent articles, e.g., sanitary napkins; catamenial pads; incontinence guards; diapers or training pants for infant, child, or adult care; bandages or wound dressings and like personal care absorbent articles.

During use of a personal care absorbent article, body exudates such as menstrual fluids, urine, or the like, will be absorbed by the absorbent core, which tends to discolor the absorbent article and makes the cover material appear wet. These conditions are generally not desirable or acceptable to the end user. Rather, end users generally prefer that the absorbent article provide a clean appearance and a dry surface after absorption of fluids.

In addition, it is generally desirable for the end user to be able to ascertain the special properties or instructions that the absorbent article may have prior to use. A lot of times, it is desirable and convenient for the end user to carry individual absorbent articles without the bulk packaging instructions or product information that is generally displayed thereon. Thus, there is a desire to have indicia printed on each individual absorbent article. However, printing on the cover layer presents difficulties. For example, because the cover layer faces the user's skin, bleeding and/or color rub off of the indicia can occur towards the user's skin, which can be exacerbated under the wet conditions associated with the discharge of bodily fluids.

Accordingly, there remains a need for improved cover materials having improved absorbent properties as well a need for indicia to be displayed on the cover layer that does not rub off or bleed during use.

### BRIEF SUMMARY

Disclosed herein, in one embodiment, is an absorbent article comprising a cover layer comprising a user facing surface and a bottommost surface and a sorbent layer disposed between the cover layer and the bottommost surface. The user facing surface has ink indicia imprinted thereon with a surfactant and/or a botanical extract.

In one embodiment, a process for forming a cover layer of an absorbent article comprises imprinting a user facing surface of a polymeric film with an ink to form indicia thereon; forming apertures in the polymeric film, wherein the apertures form a protuberance having a tapered profile extending from the user facing surface to a bottom surface; and treating the user facing surface the apertures with a surfactant and/or a botanical extract.

In one embodiment, a feminine care product comprises a cover layer containing a cover material and indicia imprinted thereon, a liquid impervious bottom layer bonded to the cover layer, and an absorbent core intermediate the cover layer and the bottom layer. The indicia is formed from an ink comprising a surfactant and/or a botanical extract in an amount effective to increase the absorptive properties of bodily exudates into the cover layer as compared to the cover layer without the surfactant and/or botanical extract.

The above described and other features are exemplified by the following detailed description and drawings.

### BRIEF DESCRIPTION OF THE DRAWINGS

FIG. 1 is a perspective cutaway view of an exemplary absorbent article in the form of a sanitary napkin for feminine care protection;

FIG. 2 is an enlarged perspective view of an apertured cover layer for the absorbent article; and

FIG. 3 is an elevational view of a vacuum aperturing apparatus.

FIG. 4 is an example of the designs that can be printed into the cover material.

FIG. 5 is an example of the designs that can be printed into the cover material.

### DETAILED DESCRIPTION

Disclosed herein is an absorbent article having a polymeric film cover layer, optionally containing a plurality of apertures, has imprinted ink indicia with surfactant(s) and/or botanical extract(s) (e.g., coated over the cover layer or included in the ink indicia). As used herein, the term “absorbent article” generally refers to devices used to absorb body exudates, and more specifically, to devices that may be placed against, in proximity to, or inside the body of the wearer to absorb and contain body exudates. As such, the term includes, without limitation, absorbent articles such as diapers, catamenial pads, tampons, sanitary napkins, incontinent pads, training pants, and the like, as well as wipes, bandages, and wound dressings.

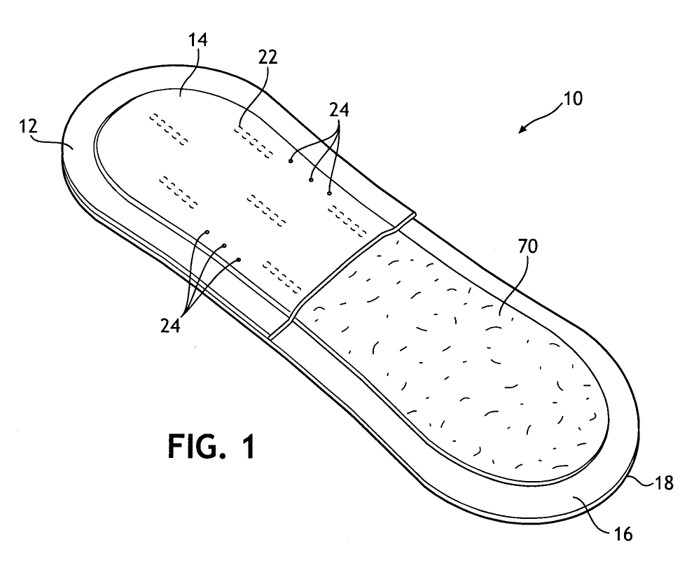

Referring now to FIG. 1, there is shown an exemplary absorbent article generally designated 10 designed for feminine care protection. The absorbent article 10 is illustrated as a sanitary napkin for feminine hygiene having generally a racetrack shape. However, the absorbent article can be a pantiliner, pantishield, or any other disposable absorbent article that is well known in the art, and can include other shapes, such as oval, hourglass, straight sided, wrapped and peripheral sealed constructions. It should also be noted that absorbent articles generally come in a variety of sizes and shapes and also vary in thickness. For example, in some embodiments, the absorbent article 10 is between about 150 millimeters (mm) to about 320 mm long, and between about 60 mm to about 120 mm wide and has a racetrack shape with rounded ends. Moreover, in some embodiments, the absorbent article has a thickness or caliper of less than about 20 mm. For example, when formed as a sanitary napkin, the absorbent article preferably has a caliper of less than about 15 mm, in some embodiments less than about 5 mm, and in some embodiments, less than about 4 mm.

The illustrated absorbent article 10 generally comprises a liquid permeable cover layer 12 having a user facing surface 14; a liquid impermeable bottom layer 16 having an outer facing surface 18; and an absorbent core 20 located intermediate between the cover and bottom layers 12, 16, respectively. As will be described herein, indicia 22 of words (e.g., aloe) and/or images are imprinted onto the cover layer 12. The cover layer 12 in one of the embodiments also contains a plurality of apertures 24-formed therethrough to permit bodily fluid to pass more readily into absorbent core 20. As shown, the cover 12 and the bottom layer 16 can extend beyond the absorbent core 20 as shown and be peripherally joined together, either entirely or partially, using known techniques, or alternatively, the cover layer 12 can surround the absorbent core 20 so that it completely encases the absorbent article 10. Typically, adhesive bonding, ultrasonic bonding, or any other suitable joining method known in the art joins the cover layer 12 to the bottom layer 16, which effectively encapsulates the absorbent core 20 therein.

The cover layer 12 is sanitary, clean in appearance, and preferably opaque to hide bodily discharges collected in and absorbed by the absorbent core 20. The cover layer 12 further exhibits good strike through and rewet characteristics permitting bodily discharges to rapidly penetrate through the cover to the absorbent core 20, but not allow the body fluid to flow back through the cover layer 12 to the skin of the wearer. The cover layer 12 generally comprises a polymeric film comprised of a single layer or, alternatively, multiple layers and/or natural or synthetic fibers.

In an exemplary embodiment, the cover layer 12 is formed of a thermoplastic polymeric material, or a blend comprising of least two immiscible polymeric film materials. A representative, non-limiting list of polymeric materials suitable for fabricating the cover layer 12 includes, but is not intended to be limited to, polyolefins, such as polypropylene and polyethylene; polyolefin copolymers, such as ethylene-vinyl acetate (“EVA”), ethylene-propylene, ethylene-acrylates, and ethylene-acrylic acid and salts thereof; halogenated polymers; polyesters and polyester copolymers; polyamides and polyamide copolymers; polyurethanes and polyurethane copolymers; polystyrenes and polystyrene copolymers; and the like. Most often, the cover layer 12 is fabricated from polyolefins, and especially polyethylene and copolymers of polypropylene and ethylene.

In the case of a cover layer 12 comprised of multiple layers, a topmost layer (having the user-facing surface 14) may extend into and/or through a bottommost layer of the multilayer and have land areas between apertures in each respective layer. The permeability of the bottommost layer for the cover layer is preferably about equal to or higher than the permeability of the top layer. In this manner, bodily fluids can readily pass into the absorbent core 20. In the same fashion, the wettability of the second layer can be higher than the one exhibited by the first layer to make this material more absorbent.

The cover layer 12 may be non-woven, woven, a laminate, foam, a film, fibrous structures, or a mixture or composites comprising at least one of the foregoing. The various forms may be prepared by a carding process, a spunbond process, or a meltblown process. A spunbond process refers to small diameter fibers that are formed by extruding molten thermoplastic material as filaments from a plurality of fine, usually circular capillaries of a spinneret with the diameter of the extruded filaments then being rapidly. Meltblown processes refer to fibers formed by extruding a molten thermoplastic material through a plurality of fine, usually circular, die capillaries as molten threads or filaments into converging high velocity, usually hot, gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Meltblown fibers are microfibers that may be continuous or discontinuous, are generally smaller than 10 micrometers in average diameter, and are generally tacky when deposited onto a collecting surface.

In order to render the cover layer 12 opaque, it may be generally desired to provide non-woven materials with a relatively high surface area. Such high surface areas may be suitably accomplished by forming the non-woven material from fibers having a denier (d) of less than or equal to about 3.0 d, alternatively of less than or equal to about 1.0 d. The non-woven materials may suitably have a surface area greater than or equal to about 0.3 square meters per gram (m²/g), alternatively greater than or equal to about 0.5 m²/g, and still further, alternatively greater than or equal to about 0.6 m²/g to less than or equal to about 1.5 m²/g. Generally, the higher the surface area, the more opaque the non-woven material may appear. The surface area of the fibrous non-woven materials may be suitably determined by the physical gas adsorption method of Bruanauer, Emmet, and Teller (B.E.T.), Journal of American Chemical Society, Vol 60, 1938, p 309, as standardized in ASTM D 4820-92a. The multi-point (5 points), static volumetric method may be used with krypton as the adsorption gas, and 90° C. as the preliminary drying temperature. Another way to improve the nonwoven opacity is the use of titanium dioxide (TiO₂) that helps to create a white filament; also pigments may be mixed in the spinning process creating fibers of different colors that can have increase opacity. In addition, the fibers may be treated with calcium carbonate that provides a white color to the filaments and also improves the tactile properties of the nonwoven material.

The cover layer 12 may also be formed from a blend of at least two immiscible thermoplastic polymeric components. The first thermoplastic polymeric component may form a continuous phase that may exhibit a first melting point temperature. In order to form the continuous phase, it may be preferred that the first thermoplastic polymeric component be present at about 45 to about 95 weight percent (wt %) of the layer, and more preferably at about 60 to about 80 weight percent of the layer, based upon the total weight of the layer. A dispersed phase may comprise a second thermoplastic polymeric component that exhibits a second melting point temperature. It may be preferred that the second thermoplastic polymeric component be present at about 5 weight percent to about 55 weight percent of the layer, and more preferably at about 20 weight percent to about 40 weight percent of the layer. In addition, the second melting point temperature is preferably less than the first melting point temperature to allow the film to be heated to a temperature between the first and second melting point temperatures, rendering the second thermoplastic polymeric component capable of forming an adhesive bond. This bond may be formed between different portions of the cover layer 12, or it may be between the cover layer 12 and another element of the absorbent article 10.

The apertures 24 formed in the cover layer 12 can be randomly or uniformly arranged through the cover layer 12 (or the individual layers forming the multilayered cover layer 12). Alternatively, the apertures 24 can be selectively confined to certain areas of the absorbent article 10, e.g., located in a narrow longitudinal band or strip within the absorbent article 10. The size, shape and number of apertures 24 can be varied depending on the desired application.

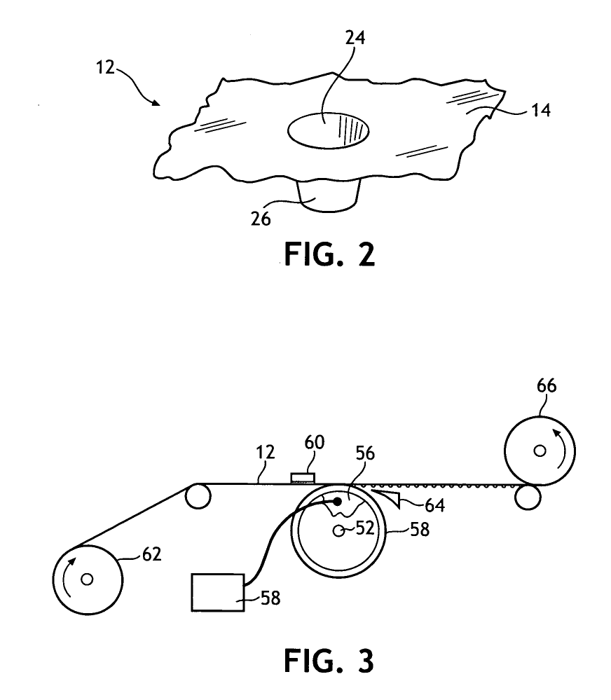

The apertures 24 may be formed in the cover layer 12 by pin aperturing, slitting and stretching of the polymeric film, or vacuum aperturing, wherein the resulting apertured cover has an open area and a plurality of protuberances 26 as shown more clearly in FIG. 2. The protuberances 26 preferably have a tapered profile. In a preferred embodiment, a vacuum aperturing process forms the apertures 24.

FIG. 3 illustrates an apparatus suitable for the vacuum aperturing process. The apparatus generally includes a rotary cylindrical drum 50 supported at each end by a centrally disposed axle 52. The outer cylindrical surface of the drum is preferably formed of high polished metal having a relatively low coefficient of friction. A molding element 54 is mounted about the drum 50. The molding element 54 may be formed as an integral unit adapted to be slipped onto the drum or it may be wrapped about the drum and secured in any suitable manner, e.g., by a vacuum exerted to the film. For purposes of rotating element 54, a gear drive may be employed on the element itself or a pulley drive may be connected to the element 54 by means of caps provided on ends thereof. A vacuum chamber 56 is positioned within drum 50 along the axis thereof and opens at the surface of the drum over a limited portion of its periphery in contact with the inner surface of molding element 54. In order to provide an effective seal of the leading and trailing edges of chamber 56, strip gaskets of rubber, graphite or other suitable material which extend slightly above the surface of drum 50 against element 54 can be used. Chamber 56 is hermetically sealed at all points except the peripheral opening on drum 10 and may be evacuated by pumping equipment 58 connected to the chamber 56 in any suitable manner. A convenient exhaust port for the chamber 56 may be provided through the central axle 52 which may open into the chamber 56 and be provided with a suitable coupling fitting on one end thereof. Immediately adjacent the opening in chamber 56 and spaced from the opposite surface of molding element 54, a heating unit 60 extends over a portion of the periphery of drum 50.

In operation, the polymeric film for forming the cover layer 12 is fed from a roll 62 onto the rotating molding element or may be first subjected to any of the preliminary treatments previously discussed above. The polymeric film preferably has indicia imprinted prior to the aperturing process. The imprinting process may be continuous or discontinuous to the aperturing process. Rotation of the molding element 54 causes the film 12 to pass beneath heater 60 where the material is heated to a softening temperature and thence over vacuum chamber 56 which causes the thermoplastic material to flow uniformly into the design perforations provided in molding element 54. The softening temperature is preferably greater than about room temperature to about 400° C., and is a function of the dwell time. In the region immediately following vacuum chamber 56, the film molded in conformity with element 54 may be subjected to a cooling or fixing operation in accordance with the physical properties of the material employed. The molded sheet might then be stripped from molding element 54 by means of an optional stripping device 64 disposed tangentially to element 14 and fed to a rotating rewind drum 66. Drum 66 is preferably provided with an idler clutch arrangement to eliminate any possible strain on sheet 12 during the rewind operation. In one embodiment, a treatment station can be located between point 64 and the unwinder to treat the apertured material. This treatment can be applied by techniques such spaying or foam that will not damage the pores created to the film. In addition, the film can be post dried to reduce the surfactant migration prior to be winded.

The open area of the cover layer 12 is defined as the area occupied by apertures 24 relative to the total area provided by the film. Cover layer 12 is preferably produced with an open area of about 5 percent to about 35 percent, and more preferably of about 10 percent to about 25 percent, wherein each aperture has a pore size greater than or equal to about 100 micrometers (μm) to about less than equal to about 700 μm equivalent circular diameter (ECD). The pore size may be uniform or non-uniform depending on the desired application.

In the case of a cover layer 12 formed of different multiple layers, each layer is preferably apertured such as by co-aperturing the top, bottom, and intermediate layers, if present. Such co-aperturing may be accomplished by a number of processes including a matched roll pin aperturing process, a pattern/anvil roll pin aperturing process, vacuum aperturing, and the like.

Alternatively, the apertured film may be obtained from a commercial vendor. Suitable apertured polymeric films may include Vispore® apertured film supplied by Tredegar. Suitable apertured films include, but are not limited to, those available commercially under the designations Tredegar X-6799, Tredegar X-6845, Tredegar X-6923, Tredegar X-6944, and Tredegar X-6844. The polymeric film may have a female side, which is smooth, and a male side, which may be somewhat less smooth, due to the protuberances surrounding the apertures. If the film has been apertured, ink jet printing or spraying can be used to print the patterns without damaging the pores in the film.

As previously discussed, prior to forming the apertures 24, the cover layer 12 is preferably first imprinted with indicia 22. The indicia may include images, instructions for use, identifiers of special properties particular to the absorbent article, brand name, visual cues, other information and/or brand identifiers, and the like. By printing indicia on or within the cover layer 12, maximum contrast can be obtained for the indicia, which enhances readability for the end user.

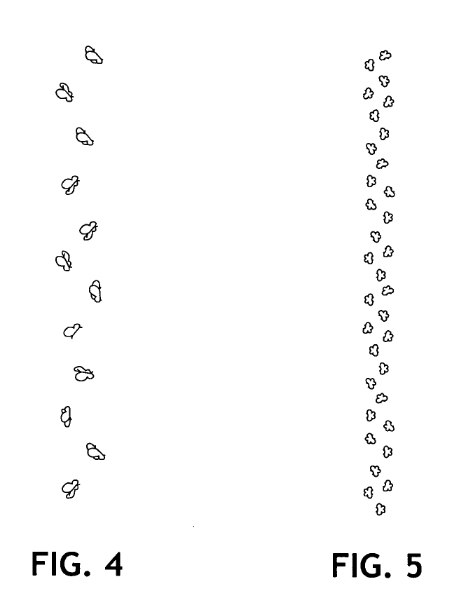

Two representative indicia designs are shown as examples in FIGS. 4 and 5 illustrating different configurations of butterflies. As will be apparent, a literally unlimited number of designs may be selected as desired for esthetic and utilitarian purposes.

In an exemplary embodiment, the polymeric film for forming the cover layer 12 is first printed on the inner and/or outer surfaces. Generally, the first layer is printed on its inner surface, the surface in face-to-face relation with a second layer as in the case of a laminated multi-layer structure or in face-to-face relation with the absorbent core 20. This construction may be preferred as it may further improve the abrasion resistance (color rub-off resistance) properties of the printed polymeric film. Alternatively, reverse printing onto an inner surface may be employed, which further reduces the risk of the ink transferring to the user.

Any printing process, for example, flexographic, rotogravure, ink jet, or a combination comprising at least one of the foregoing processes may be used. The indicia 22 may be obtained by generating a halftone. Thus, a general printing process may be halftone printing. As used herein, the term halftone means breaking up a continuous solid tone into a plurality of tiny individual indicia of varying sizes, shapes and/or tonal intensities (tonalities).

The inks used in the printing process to form the indicia are preferably particulate-type inks. The inks chosen should, of course, be safe for human use and should not have environmentally deleterious effects. It is preferred that the ink remain on the cover layer, even when wet, and not transfer to the skin of the wearer. Moreover, it is desirable that the ink be suitable for the intended printing process and is preferably temperature resistant to the process employed for forming the absorbent article, e.g., the temperatures used during a vacuum aperturing process and like elevated heating processes. An particularly preferred ink is commercially available from Sunchemical de Mexico, Alce Blanco #20, Esquina con Calle 9, Naucalpan de Juarez, C.p. 53370 Edo. De Mexico, under the family MO3Z-XXX-FF in which XXX can be: 484 (blue), 486 (green), 485 (violet), 487 (black), 489 (pink), 968 (white). Combinations of these inks can also be employed.

The particulate inks preferably comprise inert pigments and dyes, collectively referred to as pigments, which may be added in levels of about 0.25 weight percent to about 40 weight percent on a dry weight basis (based upon the total dry weight of the ink), and preferably about 1 weight percent to about 10 weight percent.

Suitable pigments include: azo dyes (e.g., Solvent Yellow 14, Dispersed Yellow 23, and Metanil Yellow), anthraquinone dyes (e.g., Solvent Red 111, Dispersed Violet 1, Solvent Blue 56, and Solvent Orange 3), xanthene dyes (e.g., Solvent Green 4, Acid Red 52, Basic Red 1, and Solvent Orange 63), azine dyes (e.g., Jet Black), and the like. Other suitable organic pigments, include: dairylide yellow AAOT (for example, Pigment Yellow 14 CI No. 21095), dairylide yellow AAOA (for example, Pigment Yellow 12 CI No. 21090), Hansa Yellow, CI Pigment Yellow 74, Phthalocyanine Blue (for example, Pigment Blue 15), lithol red (for example, Pigment Red 52:1 CI No. 15860:1), toluidine red (for example, Pigment Red 22 CI No. 12315), dioxazine violet (for example, Pigment Violet 23 CI No. 51319), phthalocyanine green (for example, Pigment Green 7 CI No. 74260), phthalocyanine blue (for example, Pigment Blue 15 CI No. 74160), naphthoic acid red (for example, Pigment Red 48:2 CI No. 15865:2), and the like. Inorganic pigments include: titanium dioxide (for example, Pigment White 6 CI No. 77891), carbon black (for example, Pigment Black 7 CI No. 77266), iron oxides (for example, red, yellow, and brown), ferric oxide black (for example, Pigment Black 11 CI No. 77499), chromium oxide (for example, green), ferric ammonium ferrocyanide (for example, blue), and the like. Additionally, combinations comprising at least one of the foregoing pigments.

In an exemplary embodiment, imprinting the ink onto the cover layer 12 comprises flexographic printing to provide the proper balance of cost effective, high speed, high quality printing suitable for printing, the polymeric film. Flexography is a printing technology that uses flexible raised rubber plates or photopolymer plates to carry the image to the film. The flexible plates generally carry a low-viscosity ink directly onto the film.

The inks are generally dispersed or dissolved in a low viscosity carrier. Exemplary solvents are aliphatic hydrocarbons with common binder types, such as polyamide, shellac, nitro-cellulose, and styrene-maleic, and the like, as well as combinations comprising at least one of the foregoing. Generally, solvent-based inks include non-catalytic, block urethane resin, which generally demonstrate superior durability over traditional flexographic binders, such as styrene-maleic, rosin-maleic, and acrylic solutions. Desired solvent blends include various acetates such as ethyl acetate, N-propyl acetate, isopropyl acetate, isobutyl acetate, N-butyl acetate, and blends comprising at least one of the foregoing; various alcohols including ethyl alcohol, isopropyl alcohol, normal propyl alcohol, and blends comprising at least one of the foregoing; and glycol ethers including Ektasolve® EP (ethylene glycol monopropyl ether), EB (ethylene glycol monobutyl ether), DM (diethylene glycol monomethyl ether), DP (diethylene glycol monopropyl ether), and PM (propylene glycol monomethyl ether), which may, for example, be obtained from Eastman Chemical, Kingsport, Tenn., as well as combinations comprising at least one of the foregoing. Other glycols that may also be used are DOWANOL® obtainable from Dow Chemical, Midland, Mich. An exemplary solvent blend may be a blend of about 50 weight percent to about 75 weight percent glycol ether, about 25 weight percent to about 35 weight percent N-propyl acetate, and about 15 weight percent to about 25 weight percent N-butyl acetate, based upon the total weight of the ink composition.

Suitable water-based inks that may be used include emulsions that may be stabilized in water-ammonia and may further comprise alcohols, glycols, or glycol ethers as co-solvents. Generally, organic solvents (e.g., in amounts of less than or equal to about 7 wt %, based upon the total weight of the emulsion, preferably about 0.25 wt % to about 7 wt % when they are employed) may be added to water-based inks. For example, alcohols (for example, propan-2-ol) may be added for speeding up drying and assisting wetting; glycols (for example, mono propylene glycol) may be added to slow down drying, and; glycol ethers (for example, dipropylene glycol mono methyl ether) may be added to aid film formation. Such solvents may be commodity chemicals, commercially available from various companies. Generally, water-based ink includes self-crosslinking acrylic copolymer emulsions that may have superior durability over non-crosslinking binders such as acrylic solutions and dispersion copolymers.

Besides the solvent and pigments, the inks may also comprise a binder. The binder helps stabilize the pigment onto the cover layer 12. Generally, the pigment-to-binder ratios is typically about 1:20 to about 1:2 and can be up to about 1:1.7.

Waxes may also be included to increase the slip and improve the rub-resistance of the inks of the printed polyolefin substrate. Common classifications of waxes include animal (for example, beeswax and lanolin), vegetable (for example, carnauba and candellilia), mineral (for example, paraffin and microcrystalline), and synthetic (for example, polyethylene, polyethylene glycol, and Teflon®). Optionally about 0.5 weight percent to about 5 weight percent wax, based on the total ink formulation weight, can be employed.

After the indicia is imprinted onto the cover layer 12 and the apertures may be formed, the cover layer 12 is preferably sprayed, immersed, slot coated, brushed, transfer coated, or otherwise coated with the surfactant and/or the botanical extract compound to enhance liquid penetration to the absorbent core 20. Moreover, the use of surfactants and/or botanicals extracts can reduce process friction, such as absorbent article press ejection forces, and may be sufficient to prevent damage to the product during manufacture. If the surfactants and/or botanicals extracts are added to the ink composition, they can comprise about 10 weight percent to about 50 weight percent of the total weight of the ink composition. If they are disposed over the ink composition that has been disposed on the cover layer (e.g., over all or a portion of the cover layer comprising the ink), the surfactant is preferably about 0.1 weight percent to about 3 weight percent of the total weight of the cover layer 12, while the botanical extracts are preferably about 0.01 weight percent to about 30 weight percent of the total weight of the cover layer 12.

The surfactants and/or botanical extracts enable the redirection of the hydrophobic properties of the water insoluble indicia 22 such that enhanced absorption of body exudates can occur in the printed areas. As a result, the use of surfactants and/or botanical extracts permits an increase in indicia density without affecting overall adsorption into the absorbent core 20. With this approach the fluid absorption is better managed since products can be designed having both less wettable and preferentially higher wettable areas, enabling the fluid to be directed towards the absorption target area. In one embodiment, this approach can be employed to modify the wettability of the ink to make it hydrophobic, and thereby creating a barrier for the fluid and helping reduce the amount of fluid running off the product.

Combinations of surfactant(s) and/or botanical extracts desirably have a hydrophilic/lyophilic balance number (HLB) of greater than or equal to about 7, more desirably greater than or equal to about 10, and even more desirably, a HLB of greater than or equal to about 14. As used herein, the term “hydrophilic agent” refers to a substance that may readily associate with water, and the term “lyophilic agent” refers to an agent that may attract liquids in a colloid system, describing a colloidal system in which the dispersed phase may be a liquid and attracts the dispersing medium. Hydrophilic agents that do not generally have a measured HLB may also be used. Such hydrophilic agents may include surface active agents(s) (or surfactants) without limitation.

The hydrophilic agents that do not generally have a measured HLB may also be used. Such hydrophilic agents may include, without limitation, diols, such as glycols and polyglycols. Suitable nonionic surfactants include, but are not intended to be limited to, C₂₋₈ diols and polyglycols, and the like. Generally, the diol may be glycols (C₂ and C₃ diols) and polyglycols. The term “polyglycol” refers to a dihydroxy ether formed by dehydration of two or more glycol molecules. A representative, non-limiting list of useful polyglycols, includes: ethylene glycol, propylene glycol, polyethylene glycols, polypropylene glycols, methoxypolyethylene glycols, polybutylene glycols, block copolymers of butylene oxide and ethylene oxide, and the like, as well as combinations comprising at least one of the foregoing.

Other suitable nonionic surfactants include: ethoxylates, including fatty acid ester ethoxylates, fatty acid ether ethoxylates, and ethoxylated sugar derivatives (e.g., ethoxylated fatty acid polyesters, ethoxylated fatty acid sorbitan esters, and the like), and the like, as well as combinations comprising at least one of the foregoing. Representative ethoxylated fatty acid sorbitan esters include: polyoxyethylene sorbitan laurate (also known as Polysorbate 20 (HLB: 16.7) and 21(HLB: 13.3)), polyoxyethylene sorbitan palmitate (also known as Polysorbate 40 (HLB: 15.6)), polyoxyethylene sorbitan stearate (also known as Polysorbate 60 (HLB: 14.9) and 61 (HLB: 9.6)), polyoxyethylene sorbitan tristearate (also known as Polysorbate 65 (HLB: 10.5)), polyoxyethylene sorbitan oleate (also known as Polysorbate 80 (HLB: 15.0) and 81 (HLB: 10.0)), polyoxyethylene sorbitan trioleate (also known as Polysorbate 85 (HLB:11.0)), and the like, as well as combinations comprising at least one of the foregoing. Among the aforementioned ethoxylated fatty acid sorbitan esters, polyoxyethylene-20-sorbitan monolaurate is generally preferred.

Another generally used class of ethoxylated fatty acid ethers may be the class of polyoxyethylene alkyl ether. A representative, non-limiting, list of useful polyoxyethylene alkyl ethers, includes polyoxyethylene lauryl ether, polyoxyethylene stearyl ether (also known as Steareth-2, Steareth-10 (HLB: 12.4), and the like), polyoxyethylene cetyl ether (also known as Ceteth-2, Ceteth-10 (HLB: 12.9), and the like), and polyoxyethylene oleyl ether (also known as Oleth-2 (HLB: 12.4), Oleth-10, and the like), as well as combinations comprising at least one of the foregoing. Among the aforementioned polyoxyethylene alkyl ethers, polyoxyethylene stearyl ether is most generally preferred.

Another generally used class of fatty acid esters may be the class of sorbitan fatty acid esters. A representative, non-limiting, list of useful sorbitan fatty acid esters, includes: sorbitan monooleate (HLB: 4.3), sorbitan monostearate (HLB: 4.7), sorbitan monopalmitate (HLB: 6.7), sorbitan monolaurate (HLB: 8.6), sorbitan tristearate (HLB: 2.1), sorbitan trioleate (HLB: 1.8), as well as combinations comprising at least one of the foregoing. Among the aforementioned sorbitan fatty acid esters, sorbitan monooleate is the most generally preferred.

Another generally used class of ethoxylated sugar derivatives may be the class of methyl glucose derivatives. A representative, non-limiting list of useful methyl glucose derivatives, includes: methyl glucose-10, methyl glucose-20, methyl glucose-20 distearate, methyl glucose dioleate (HLB: 5), methyl glucose sesquistearate (HLB: 6), PEG-120 methyl glucose dioleate, PEG-20 methyl glucose sesquistearate, as well as combinations comprising at least one of the foregoing.

Suitable surfactant combinations that are commercially available include those marketed under the registered trademarks “SPAN” (sorbitan derivatives), “TWEEN” (polysorbate derivatives), and “BRIJ” (polyoxyethylene oleyl ethers) by Uniqema, a division of ICI, Wilmington, Del., USA and those surfactants marketed under the registered trademarks “GLUCAM” (methyl glucose ethers), “GLUCATE” (methyl glucose derivatives), and “GLUCAMATE” (polyethyleneglycol ethers of methyl glucoses) by Amerchol Corporation, Edison, N.J., USA.

Suitable botanical extracts include, but are not limited to, chamomile, aloe vera, jojoba, sunflower oil, citric oils, carrot oil, avocado oil, almond oil, cotton extract, vitamin extracts, and the like, as well as combinations comprising at least one of the foregoing. The polymeric film cover layer 12 may also be treated with vitamin extracts such as vitamin E, and the like.

It is noted that the surfactant(s) and botanical extract(s) may be used individually or in combinations comprising at least one of the foregoing surfactants and/or botanical extracts. Additionally, they may be disposed in the ink or may be a coating on all or a portion of the cover layer.

In addition, other components and additives may be added to the cover layer 12 in an amount that may not hinder obtaining the object of the present disclosure, including, without limitation, antioxidants, UV absorbers, lubricants, antiblock and slip agents, plasticizers, nucleating agents, antistatic agents, flame retardants, pigments, dyes, inorganic or organic fillers, as well as combinations comprising at least one of the foregoing. The cover layer 12 may, if desired, comprise Triclosan, or a like anti-bacterial agent in an anti-bacterially effective amount.

The absorbent core 20 may be made from various materials including rayon fibers; natural fibers, such as, cotton fibers and wood pulp fibers; synthetic fibers, such as, polyester fibers, polyamide fibers, polyolefin fibers; and the like, as well as combinations comprising at least one of the foregoing. The fibers may be bicomponent fibers such as, for example, a sheath-core configuration in which the sheath comprises one polymer and the core comprises a different polymer. Bicomponent fibers having other configurations, such as, for example, a side-by-side configuration, may also be used. Also the addition of superabsorbent materials can be used to enhance the absorption capacity of the absorbent system.

Generally, the fibers comprising the absorbent core 20 may be bonded at contact points where the fibers cross. The bonding may be achieved, for example, by heating the fibers so that they soften and fuse together at their crossover points. Alternatively, the fibers may be bonded by the use of an adhesive that may be applied by, for example, spraying or gravure printing methods. Generally, the fibers may be solid fibers; or, the fibers, or portions thereof, may be hollow fibers. Fibers that may be used for the absorbent core 20 preferably comprise deniers of about 3 d to about 10 d. The basis weight for the absorbent core is not intended to be limited, and generally is about 0.003 grams per square centimeter (g/cm²) to about 0.015 g/cm².

The structure of the absorbent core 20 may be manufactured in a wide variety of sizes and shapes and from a wide variety of liquid-absorbing materials. A representative, non-limiting list of useful materials includes: cellulosic materials (such as rayon, cotton, wood pulp, creped cellulose wadding, tissue wraps and laminates, peat moss, and chemically stiffened, modified, or cross-linked cellulosic fibers); synthetic materials, (such as polyester fibers, polyolefin fibers, absorbent foams, absorbent sponges, super-absorbent polymers, absorbent gelling materials); formed fibers, (such as capillary channel fibers and multi-limbed fibers); and the like, as well as combinations comprising at least one of the foregoing, such as synthetic fibers and wood pulp including co-formed fibrous structures. The absorbent core 20 may further include transfer members, intake members, surge layers, and the like.

As previously described, the bottom layer 16 is generally liquid impermeable and has an outer facing surface. The bottom layer 16, in some cases, could permit the passage of air and other vapors through the absorbent article while blocking the passage of bodily fluids. Any liquid impermeable material can generally be used to form the bottom layer 16. For example, one suitable material that can be utilized is a microembossed polymeric film such as polyethylene or polypropylene. In particular embodiments, a polyethylene film has a thickness of about 0.2 mils (about 5.1 micrometers) to about 5 mils (about 127 micrometers), and more particularly, about 0.5 mils (about 12.7 micrometers) to about 3 mils (about 76.2 micrometers).

In addition, other components and additives as described above may be added to the polymeric film material in an amount that may not hinder obtaining the object of the present disclosure.

The product can also have a secondary layer “underlayer” that is placed beneath the cover layer 12. This material can be made from a thermally bonded carded web process, thru air bonded process, or woven material. Fibers used in these processes include polyolefin fibers, natural fibers, bicomponent fibers, and the like, as well as combinations comprising at least one of the foregoing. The underlayer could comprise higher permeability and/or higher wettability than the body side cover material to improve the absorption behavior of the absorbent system. If desired, this layer may also have botanical extracts, and may have a printed pigmented, e.g., to enhance the visual appearance of the total product.

The product can be used for multiple purposes, e.g., in the areas of feminine care and/or infant care products (e.g., as a liner). It can be printed with indicia, enhancing the visual aesthetic. For this type of printing, inks are preferably chosen in a way that will not rub off or become diminished by friction, body heat, or contact with fluids (human, or surfactant and/or botanical extract). The visual indicia can serve many purposes, among them, it can selectively apply an additive to enhance the material absorbency performance or/and have a botanical extracts or skin wellness treatment. Two representative indicia designs are shown as examples in FIGS. 4 and 5 illustrating different configurations of butterflies. As will be apparent, a literally unlimited number of designs may be selected as desired for esthetic and utilitarian purposes.

In one embodiment, the ink can be mixed with a surfactant in order to selectively modify the wettability of the surface. The wettability of the surface can the be hydrophilic (water contact angle less than 90 degrees) whenever the ink is applied in a manner that is expected to help the absorption behavior of the material, or in another case, when the design is applied to a product's sides, the product developer might choose to have a non-wettable area to prevent fluid movement outside the periphery of the pad. Depending on the process and its sophistication, it can be decided that a specific color in a design might be more wettable than another, allowing preferentially wettable areas and other areas that are hydrophobic.

After the ink has been printed, a cover material can be treated to further improve its absorption. This can be accomplished by different methods, including: corona treatment and/or topical application of a surfactant and/or a botanical extract.

Another use is the manufacture of a wipe material that is treated with surfactants to absorb different fluids and that also has printed designs that help to differentiate it from a competitor's material as well as increase brand recognition by consumers. This can also be combined with embossing designs and any other mechanical modifications that improve the material properties.

Indicia can be printed on each individual absorbent article e.g., providing usage instructions, describing the proper way that the product needs to be worn (e.g., in the case of a non-symmetric product), and or disseminating other information. This cover layer comprising the ink indicia and surfactant(s) and/or botanical extract(s) is particularly useful on feminine care products (e.g., sanitary napkins, and the like). For example, a feminine care product can comprise a cover layer containing an optional plurality of apertures and indicia imprinted thereon, a liquid impervious bottom layer bonded to the cover layer; and a sorbent core (e.g., an absorbent material core) intermediate the cover layer and the bottom layer. The indicia can be formed from an ink comprising a surfactant and/or a botanical extract in an amount effective to increase the absorptive properties of bodily exudates into the cover layer as compared to a cover layer without surfactant and/or botanical extract thereon. The surfactant(s) and/or botanical extract(s) can be disposed in the ink or can be disposed as a coating over all or a portion of the cover layer.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the invention scope thereof. It is therefore intended that the invention not be limited to the particular embodiment disclosed as the best mode contemplated for carrying out this invention, but that the invention will include all embodiments falling within the scope of the appended claims.

1. An absorbent article, comprising:
a cover layer comprising a user facing surface and a bottommost surface, wherein the user facing surface has ink indicia imprinted thereon with surfactant and/or a botanical extract; and
a sorbent layer disposed between the cover layer and the bottommost surface.

2. The absorbent article of claim 1 that comprises a cover material that is apertured by a method of vacuum aperturing, pin aperturing, hydroentanglement, ultrasonic and a combination thereof.

3. The absorbent article of claim 2, wherein the apertured cover layer comprises a tapered opening surrounding each aperture and extending from the user-facing surface to the bottommost surface.

4. The absorbent article of claim 1, wherein the indicia comprises an ink that has a second surfactant.

5. The absorbent article of claim 1, wherein the indicia is comprises an ink that has a second botanical extract.

6. The absorbent article of claim 1, wherein the surfactants are at about 0.1 weight percent to about 3 weight percent of a total weight of the cover layer.

7. The absorbent article of claim 1, wherein the botanical extracts are at about 0.01 weight percent to about 30 weight percent of a total weight of the cover layer.

8. The absorbent article of claim 1, wherein the cover layer comprises a polyolefin.

9. The absorbent article of claim 1, wherein the surfactant comprise a hydrophilic-lyophilic balance number greater than or equal to about 10.

10. The absorbent article of claim 2, wherein the apertured cover layer has an open area of about 5 percent to about 35 percent relative to the total area of the cover layer.

11. The absorbent article of claim 1, wherein the surfactant is non-ionic.

12. The absorbent article of claim 1, wherein the botanical extracts are selected from the group consisting of chamomile, aloe vera, jojoba, sunflower oil, citric oils, carrot oil, avocado oil, almond oil, cotton extract, vitamin extracts, and a combination comprising at least one of the foregoing extracts.

13. The absorbent article of claim 1, wherein the cover layer has been macroembossed.

14. The absorbent article of claim 1, wherein the cover layer is bonded to an under layer comprising a material selected from the group consisting of a thermally carded web, thru air bonded web, spunlace material, apertured film, foam, and a combination comprising at least one of the foregoing materials.

15. The absorbent article of claim 1, wherein the cover layer is bonded to an under layer that is treated with a surfactant and/or a botanical extract.

16. The absorbent article of claim 1, wherein the ink indicia comprises a particulate-type ink.

17. A process for forming a cover layer of an absorbent article, comprising:
imprinting a user facing surface of a polymeric film with an ink to form indicia thereon;
forming apertures in the polymeric film, wherein the apertures form a protuberance having a tapered profile extending from the user facing surface to a bottom surface; and
treating the user facing surface with a surfactant and/or a botanical extract.

18. The process of claim 17, wherein the polymeric film comprises a polyolefin.

19. The process of claim 17, wherein forming apertures in the cover layer further comprises a vacuum aperturing process comprising:
heating a polymeric film to a softening temperature of less than about 400° C;
feeding the heated film onto a molding sheet, wherein the molding element comprises a plurality of perforations in fluid communication with a pump;
applying a vacuum between the molding element and the polymeric film causing portions of the polymeric film to flow into the plurality of perforations in the molding element to form the apertures; and
cooling the film.

20. The process of claim 17, wherein the botanical extracts are selected from the group consisting of chamomile, aloe vera, jojoba, sunflower oil, citric oils, carrot oil, avocado oil, almond oil, cotton extract, vitamin extracts, and a combination comprising at least one of the foregoing extracts.

21. The process of claim 17, wherein the surfactant is present in an amount of about 0.1 weight percent to about 3 weight percent of a total weight of the polymeric film, and the botanical extract is present in an amount of in about 0.01 weight percent to about 30 weight percent of a total weight of the polymeric film.

22. A feminine care product, comprising:
a cover layer containing a cover material and indicia imprinted thereon, wherein the indicia is formed from an ink comprising a surfactant and/or a botanical extract in an amount effective to increase the absorptive properties of bodily exudates into the cover layer as compared to the cover layer without the surfactant and/or botanical extract;
a liquid impervious bottom layer bonded to the cover layer; and
a sorbent core intermediate the cover layer and the bottom layer.

23. The feminine care product of claim 22, further comprising the surfactant at an amount of about 0.1 to about 3 weight percent based on the total weight of the cover layer.

24. The feminine care product of claim 22, further comprising the botanical comprises at an amount of about 0.01 to about 30 weight percent based on the total weight of the cover layer.

25. The feminine care product of claim 22, wherein the surfactant is non-ionic.

26. The feminine care product of claim 22, wherein the ink indicia comprises a particulate-type ink.
